(19) United States
(12) Patent Application Publication
(10) Pub. No.: US 2024/0153624 A1
(43) Pub. Date: May 9, 2024

(54) METHOD FOR EVALUATING ELECTRICALLY CONDUCTIVE OBJECTS

(71) Applicant: MR COMP GmbH, Gelsenkirchen (DE)

(72) Inventors: Manuel MURBACH, Zürich (CH); Thomas DOERING, Essen (DE); Gregor SCHAEFERS, Bottrop (DE)

(73) Assignee: MR COMP GmbH, Gelsenkirchen (DE)

(21) Appl. No.: 18/272,365

(22) PCT Filed: Jan. 14, 2022
(86) PCT No.: PCT/EP2022/050803

§ 371 Date: Jan. 30, 2024

(30) Foreign Application Priority Data
Jan. 14, 2021 (DE) 10 2021 100 687.2

(51) Int. Cl. G16H 40/63; A61B 5/00; A61B 5/055; G01R 33/54

(52) U.S. Cl. CPC G16H 40/63; A61B 5/055; A61B 5/4851; G01R 33/543

(57) ABSTRACT

The disclosure relates to a method for evaluating electrically conductive objects comprising the steps of: (A) providing a computer-generated model of the object to be evaluated; (B) defining the one-dimensional representation of the object by means of a curve line, “center trajectory”, along the longitudinal axis of the object; (C) defining a number of radii, “blue disks”, along the center trajectory, said radii enclosing the three-dimensional geometry of the object; (D) generating a plurality of auxiliary trajectories using the radii defined in step (C), wherein the totality of the trajectories display the object with respect to its potential to absorb the applied tangential electric field, E-field; and (E) evaluating the tangential E-field at each of the trajectories generated in step (D) and the subsequent statistical preparation.

The invention relates to a numerical method for evaluating electrically conductive objects with respect to their safety when used in an electromagnetic field (E-field) environment, in particular in connection with MR examinations, the method being intended, inter alia, for improving the safety of the evaluated objects and for controlling MR devices when using the evaluated objects.

Whenever an electrically conductive or ferromagnetic object is in an electromagnetic field, this field acts on the object. In everyday life, we hardly notice the many E-fields that are increasingly present in our immediate environment, for example, due to increasing electromobility.

Indeed, at least above a certain strength, E-fields have a non-negligible influence on their environment. This influence can have consequences for the safety of people and their health, for example, if these people are carriers of ferromagnetic or conductive objects such as implants. Both active and passive implants can be influenced in their function by E-fields.

Known sources of artificial E-fields are manifold and can be roughly divided into low-frequency or static E-fields and high-frequency E-fields.

Low-frequency E-fields in the range of 0.1 Hz to 30 kHz, for example, emanate from the power supply and can be found in every household, in remote power transmission, wireless charging, e-mobility or even in the overhead lines of the railroad.

High-frequency E-fields in the range of 30 kHz to 300 GHz are known mainly in connection with radio transmissions, i.e. from broadcasting, mobile radio, but also from microwaves and medical examination devices such as a magnetic resonance tomograhps (MR) for magnetic resonance tomography (MRT).

During an MR examination, the patient is exposed to both a strong static magnetic field and a strong high-frequency E-field. In this respect, the application of the inventive method in connection with MR examinations will be discussed below by way of example, even though the inventive method is suitable for describing the influence of any E-fields on a wide variety of electrically conductive or ferromagnetic objects.

Magnetic resonance tomography is an imaging technique used primarily in medicine. In addition to diagnostic applications, MRT is also increasingly finding its supportive use during invasive interventions. This use during ongoing interventions is made possible by constantly improving imaging techniques that can provide the surgeon with real-time images of the intervention.

This also means that in the future a large number of instruments will have to be evaluated with regard to their suitability for use in connection with MR examinations or MRT-assisted interventions than has been the case to date. In particular, the instruments to be used during interventions should be mentioned here.

The advantages of MRT over other known imaging modalities, such as computed tomography (CT) or even ultrasound (US), are on the one hand the lack of radiation exposure, such as X-rays in CT, and on the other hand the high resolution of soft tissue compared to US methods.

Accordingly, more extensive use of MRT is certainly desirable.

In a simplified description, three magnetic fields in particular interact with each other in a magnetic resonance tomograph: a strong static magnetic field (Bo) in the longitudinal direction, a high-frequency alternating magnetic field (Bi, also radiofrequency or HF- or RF field) in the transverse direction, and a gradient field (dB/dt).

The static magnetic field of tomographs commonly used in hospitals has a strength of about 0.25 tesla up to 3 Tesla. Tomographs with a higher magnetic field strength of up to 7 Tesla and above are being tested.

The alternating magnetic field oscillates in the radio frequency range and essentially has the task of tilting the hydrogen nuclei, which are aligned in the longitudinal direction by the static magnetic field, into a transverse alignment.

The gradient field allows a three-dimensional spatial coding of the resonance signals.

All of the magnetic fields mentioned have an effect not only on the object being examined, for example the patient, but also on any objects placed in or on the object being examined. The effect of the magnetic fields is not limited to the tube of the tomograph, but includes to a not inconsiderable extent at least the immediate surroundings of the tomograph and thus in particular the shielded cabin, the RF-shielded examination room.

The static magnetic field, which is usually up to 3 Tesla in strength in everyday clinical use, is capable of attracting ferromagnetic materials with extraordinary force. This field acts inside and outside the tube of the MR and can lead to dislocations of ferromagnetic objects during an examination.

In addition to the static magnetic field, the alternating magnetic fields of the RF field and the gradient field act on electrically conductive materials. This affects objects in the vicinity of the MR as well as objects inside the MR bore.

In particular, the high-frequency alternating magnetic field induces currents in electrically conductive materials, which in turn can lead to heating of these objects. In relation to electrically conductive implants made of metal, for example, this means that their possible heating during an MR examination can become a non-negligible health risk for the implant wearer if the heating exceeds a critical value of approximately 4° C.

Elongated, electrically conductive objects in particular function like antennas. Electrical currents can be induced in them by the alternating magnetic fields during an MR examination, which lead to heating, especially at the ends of these objects. In particular, wires and wire-shaped parts of implants or objects located on or in the patient during the MR examination, such as bone fixators, catheters, instrument cables, electrodes, etc., can endanger the safety of the patient during the examination.

Accordingly, it is important to evaluate both MR safety and MR compatibility of objects such as implants, as well as any object that is within the effective range of an MR.

MR compatibility essentially concerns the property of an object to negatively influence the imaging quality of the MR. It therefore plays no role in direct safety, but can lead to misinterpretation of the images or render them unusable by lowering the image quality and creating artifacts.

MR safety relates specifically to the safety of the patient and of persons in the vicinity of the MR. The evaluation of MR safety thus serves to be able to assess possible health risks for people during an MR examination in advance.

Even though the MR safety of implants and, in particular, passive implants is discussed here, the evaluation of MR safety and the application of the method according to the invention is by no means limited to implants. Rather, the evaluation also includes, as previously mentioned, objects and instruments that are used in the context of interventional or diagnostic procedures in the course of an MR examination.

Various standards are currently known for assessing the MR-safety of an object, for example IEC 62570/ASTM F2503, ASTM F2182 and ISO/TS 10974:2018(E). Accordingly, an object examined according to the known specifications can be classified as “MR-safe”, “conditionally MR-safe” or as “MR-unsafe”.

However, the known standards have their weak points. On the one hand, these relate to the applicability of known standards for the evaluation of objects that do not have typical antenna properties, i.e. that in particular do not have an elongated shape but rather have a voluminous three-dimensional shape.

Even with the required transfer of model measurements to the values actually expected in vivo, the application of known standards leads to overconservative results, especially for such objects, which excludes an excessive number of objects from use in connection with MRT examinations, since the approach there predicts, for example, too high heating. For voluminous 3D structures, it is difficult to determine an unambiguous transfer function, since these cannot be mapped as easily electro-technically as, for example, electrodes can.

Furthermore, no methods are known so far that are suitable for evaluating the safety of objects in arbitrary E-fields. As mentioned above, not only the safety of objects with respect to their exposure to E-fields in clinical contexts is of increasing interest. Rather, a large number of objects are also and increasingly exposed to E-fields in normal everyday life, which can influence a safe function or the safety of these objects in general.

The power that a given object will actually absorb during an MRT examination and the degree to which it will heat up accordingly depends on a variety of parameters and is extremely difficult to calculate in advance based on models, because the parameters to be considered when evaluating MR safety involve both the implant and the patient, as well as the MRT or MRT sequence(s) used in the particular examination.

On the implant side, for example, the dimensions and materials of the implant must be considered.

On the patient's side, the physical constitution and, in particular, the proportion of fatty tissue must be taken into account.

In the interaction between implant and patient, the exact position of the implant in the body must be taken into account.

Finally, with regard to the MRT, the specific field strengths and, in particular, the specific MRT sequences must be taken into account, which have a very decisive influence on the excitation of the implant, the induction of currents and thus the heating. Here, differences can be found not only between the various manufacturers and models of MRTs but also between individual devices.

Finally, the exact position of the patient and thus the exact position of the implant in the MR bore must be taken into account, because the magnetic field or electromagnetic field is not homogeneous within the MR bore.

The non-standardized but as previously described required transfer of the model results to the real situation during an MRT examination is correspondingly complicated and leads to the fact that a multitude of individual non-standardized procedures exist, each of which must be checked for their correctness.

It is the task of the present invention to provide a numerical method which is improved compared to the state of the art and with which the safety of an electrically conductive object in general and, in particular, the MR safety of an electrically conductive object can be determined as reliably as possible in the model. The theoretical prediction should be as close as possible to the behavior of the object to be expected in practice and thus not make any unreasonably high demands on safety on the one hand and not set any thresholds that are too low on the other.

This task is solved by a process having the features of claim 1. Advantageous embodiments are in each case the subject of the dependent claims. It should be pointed out that the features listed individually in the claims can also be combined with one another in any desired and technologically useful manner and thus show further embodiments of the invention.

The method according to the invention, which may be computer-implemented, simulates the behavior of an electrically conductive object in an electromagnetic field and is suitable for enabling both technical optimizations of the objects themselves and controls of electromagnetic field-generating devices, for example MR devices.

The method according to the invention comprises the following steps:
(A) Providing a computer-generated model of the object being evaluated.

The model should preferably be available as a CAD (computer-aided design) file. The three-dimensional geometry of the object is known.
(B) Defining the one-dimensional representation of the object by means of a curve line (center trajectory) along the longitudinal axis of the object.

The center trajectory preferably runs between the two most distant points and through the axial center of the object. If there are several exposed end points, this can be several curve lines.
(C) Defining a number of radii (blue disks) along the center trajectory, said radii enclosing the three-dimensional geometry of the object.

Preferably, at least two radii are provided, with at least a first radius provided at a first end of the object and a second radius provided at a second end of the object.

Depending on the complexity of the object, the distances between the blue disks should be chosen shorter or longer. For simple rectilinear objects, 2-3 blue disks are sufficient.

At narrow places of the object the radius is to be chosen correspondingly smaller, at voluminous places of the object the radius is to be chosen correspondingly larger, so that the object is overhung at every point.

The radii can be additionally enlarged, for example according to the expected freedom of movement, if the object is, for example, an implant within the body of a patient. The radii should then at least completely encompass the object and, in particular in the case of movable objects, take into account possible placement uncertainty of the object.

For more complex objects, i.e. objects that are not linear but, for example, curved or branched, the distance between the blue disks should not be smaller than the radius of adjacent blue disks themselves in order to keep possible angles between auxiliary trajectories and center trajectory small.
(D) Generating a plurality of auxiliary trajectories using the radii defined in step
(C). The number of generated auxiliary trajectories depends in particular on the complexity of the object.

The auxiliary trajectories each originate in the first blue disk and end in the last blue disk, preferably passing all blue disks in between.

By gradually increasing the number, statistical convergence can be estimated and brought up to the desired value. Typically, 100 to 1000 auxiliary trajectories are required for an object of average complexity. Less complex objects usually require a low number of auxiliary trajectories, complex objects sometimes a higher number.

Provided the object is a fully implanted object such as a hip implant, the selected trajectories should not leave the assumed body tissue.

The set of trajectories maps the object in terms of its potential to absorb the applied tangential electric field (E-field).
(E) Evaluating the tangential E-field at each of the auxiliary trajectories generated in step (D) and subsequent statistical preparation.

For example, the statistics include the E-field averaged over the individual trajectories, as well as the histographic distribution over the entirety of the trajectories.

The E-field values determined in this way can be used as a realistic mean E-field, which is then used in experimental measurement studies, for example, with regard to determining the expected heating of the implant during MR examinations and for evaluating MR safety in general.

According to the invention, a computer-generated model of the object as required in step (A) means any representation of the object that is available in digital form
for example, also in a database that identifies certain coordinates of the object for further analysis.

The elevation of the model is also not decisive for the inventive concept. Thus, for certain objects, it may be advantageous to create the computer model by means of 3D scanning.

It is not decisive for the invention whether the generated data or coordinates are actually converted back into a visual 3D model of the object or whether the data or coordinates are fed to a further evaluation and calculation purely numerically.

Accordingly, models which are available by means of a 3D scanner or in the form of a pure value table are also understood as computer-generated models according to the invention.

Accordingly, a visualization of the method steps is basically not necessary and ultimately serves reasons of clarity.

In an alternative process, an evaluation can also be performed according to the steps outlined below:
(α) Create an electromagnetic model of the object to be evaluated and determine an appropriate confidence interval.

Possible methods for building the model may include analytical, numerical, or experimental procedures.

The model is used to specify the power consumption to be expected locally in vivo or the resulting temperature increase in the clinically relevant areas of the object, depending on the—if applicable—stepwise applied E-field.
(β) Validation of the model from (α) in (artificial) homogeneous media.
(γ) Determine the in vivo incident tangential electric fields in terms of their strength and phase in the clinically relevant areas, dividing these areas into sections for averaging the values collected.

The division of the ranges, i.e. the choice of range sections for averaging, must be justified.

(δ) Calculate the distribution of the area-wise power consumption and temperature rise in vivo, respectively, for the clinically relevant areas assuming the possible tangential E-fields using the validated model according to step 1.

The resulting distribution indicates the power consumption or heating of the object to be expected in vivo.

The method according to the invention and the described method variant are suitable for the evaluation of a large number of objects in a wide variety of E-fields.

The objects to be evaluated can be classified as follows, at least in the clinical area, both in terms of their placement in the MR area and in terms of their type of interaction or contact with the patient.

Regarding the placement of the objects, in particular four possible areas (0 to III) in the environment of the MR can be distinguished:
Area 0: This area is outside the RF shielded chamber.
Area I: This area is inside the RF shielded chamber but still outside the currently defined 0.5 mT line.
Area II: This area is within the currently defined 0.5 mT line but without significant influence from the RF or gradient field.
Area III: This area is located within or in close proximity to the MRT tube.

In terms of their nature of interaction and contact with the patient, objects can be further divided into:
a) Objects without skin contact with the patient;
b) Objects in contact with the skin or partially inserted objects;
c) Objects that are fully implanted.

From these classifications, the following six categories can be derived for the type of objects to be evaluated, which can of course be expanded to include additional categories for further specification. Some examples are given for each of the proposed categories.
Category i: Accessories without patient contact, such as shelves, trays, displays, fire extinguishers, etc.
Category ii: Accessories with patient contact but outside, such as wheelchairs, rolators, hypodermic needles, etc.
Category iii: Active medical devices, whether or not implanted, within the MR examination room but outside the MR bore, such as pacemakers, defibrillators, etc.
Category iv: Active instruments within the MR bore but without contact with the patient.
Category v: Instruments inside the tube in contact with the patient, such as ECG electrodes, invasive measurement catheters, etc.
Category vi: Active and passive implants within the tube such as orthopedic implants, pacemakers, defibrillators, etc.

Preferably, the method according to the invention is applicable in the evaluation of the possible heating of short passive implants, for example stents or orthopedic implants such as hip or knee implants, among others.

Due to their electrical conductivity and elongated shape, such implants also function like antennas. The currents induced by the alternating magnetic fields lead to heating of the material, especially at the ends of these implants, which can cause severe damage to the surrounding tissue.

However, the method according to the invention is also applicable for the evaluation of other electrically conductive objects which are exposed to the effective range of any E-field. The method is thus applicable to the evaluation of interactions of electrically conductive objects and E-fields, where the object can be assigned to any of the previously described categories i to vi and where the E-field is not generated by an MRT, but has another artificial or even natural origin.

In particular, the method according to the invention may find application in improving the MR safety of implants and in controlling MR devices.

Thus, the method according to the invention can be used to evaluate the possible heating of implants, for example short passive implants, such as stents, or orthopedic implants, such as hip joint prostheses or knee prostheses, based on the determined E-field values.

The possible heating of an implant depends in particular on the strength of the E-field to which the implant is specifically exposed and decisively also on the “ability” of the implant to act as an antenna and to convert the acting E-field into heat.

Accordingly, a measure against excessive heating of an implant in the E-field can be not only the reduction of the E-field itself but also the reduction of the antenna properties of the implant.

Accordingly, data on the heating of the implant can be generated in a first step from the data on E-field exposure obtained by means of the method according to the invention. If the determined heating exceeds a previously defined limit value, the determined heating no longer corresponds to the heating that can be tolerated up to the limit value. The implant must be revised.

By specifying several limit values, it is also possible to derive from the calculated heating whether this heating requires no revision, a complete revision or only a partial revision of the implant.

Repeated testing of alternative implant geometries can then be used to develop alternative implant shapes that can experience lower heating in the determined E-field in iterative testing. Provided that one of the iterative tests indicates a heating for the implant that is below the established limit, this implant can be considered safe with regard to the established limits.

A corresponding optimization or revision can be aimed at for the entire implant or only for the determined heating peaks. Accordingly, limit values can be defined for the entire implant or only for specific implant regions.

Corresponding further development is therefore possible by repeated adaptation of the implant geometry and respective evaluation with the method according to the invention, whereby an approximation of the implant shaping to a geometry optimized from the point of view of MR safety can be achieved by comparing the results.

Thus, a further process step (F1) after step (E) may be:
(F1) Determining the expected heating of the object evaluated according to steps (A) through (E) and comparing the determined heating to a previously determined heating limit.

Furthermore, the data obtained by the method according to the invention can also be used to control MR devices when examining patients, provided that the patients wear an implant evaluated by the method according to the invention.

Thus, based on the data obtained by means of the method according to the invention, it is possible to control an MR device during an MR examination in such a way that critical E-field strengths and thus, in particular, dangerous heating of the implant are avoided. For example, it is conceivable to limit an MR examination only to sequences that do not generate E-fields that produce heating that is dangerous for the implant and thus for patient safety.

Thus, alternatively, a further process step (F2) after step (E) may be:
(F2) Using the data according to steps (A) to (E) to control an MR device, wherein the control only permits those operating modes (sequences) of the MR device that generate an E-field that does not reach a critical strength and, in particular, does not cause dangerous heating of the implant.

The control has to be calculated and adjusted individually for each MR device based on the data obtained by the method according to the invention, since the highly individual performance data of MR devices do not allow a generalized control.

It would be conceivable here, for example, to set up a database on which the correspondingly created control software is made available for download. In this case, control software adapted to the corresponding MR devices can be offered for each of the evaluated implants.

Even though the method according to the invention and the possible applications have been described in particular for medical implants in connection with MR examinations, the application of the method according to the invention and the further application of the data thus obtained are not to be limited either to medical implants or to MR examinations.

Rather, the method according to the invention is suitable for evaluating any object with respect to its E-field exposure and further using the generated data, for example, to determine the safety of the object in certain E-fields, to optimize the object when used in certain E-fields, or to control corresponding E-field producing devices.

Further, the invention comprises a computer program executable on a computer, the computer program comprising instructions for carrying out the method of the invention.

The invention and the technical environment are explained in more detail below with reference to the figures. It should be noted that the figures show a particularly preferred embodiment variant of the invention. However, the invention is not limited to the embodiment variant shown. In particular, the invention encompasses, to the extent that it is technically useful, any combination of the technical features listed in the claims or described in the description as relevant to the invention.

It show:

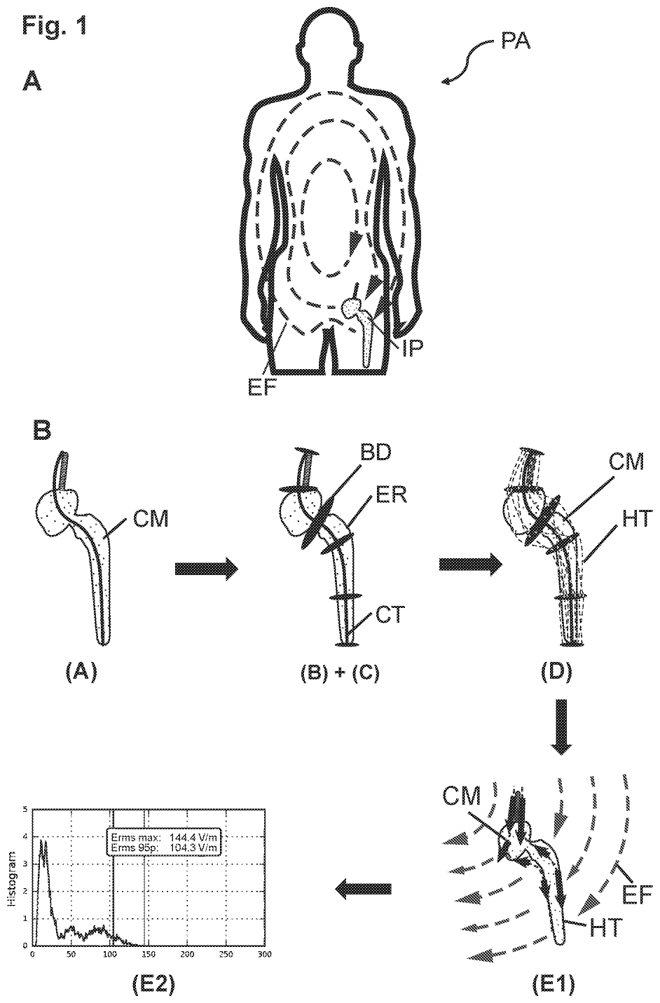

FIG. 1 The process of the method according to the invention

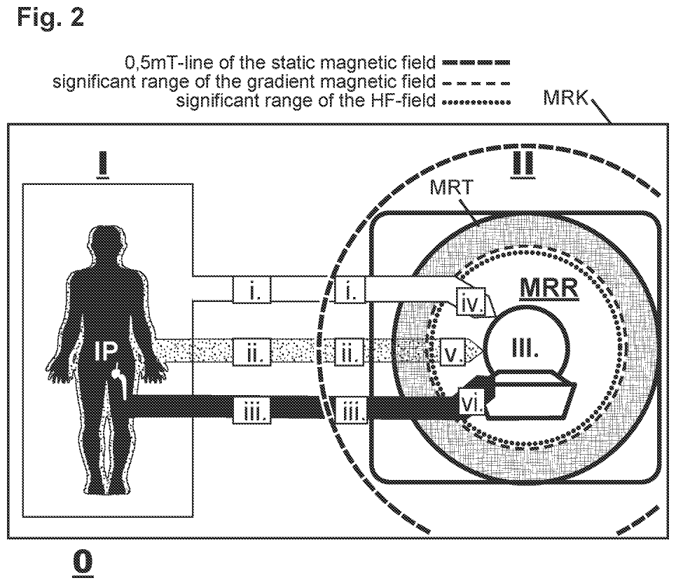

FIG. 2 The classification of objects in the vicinity of an MRT

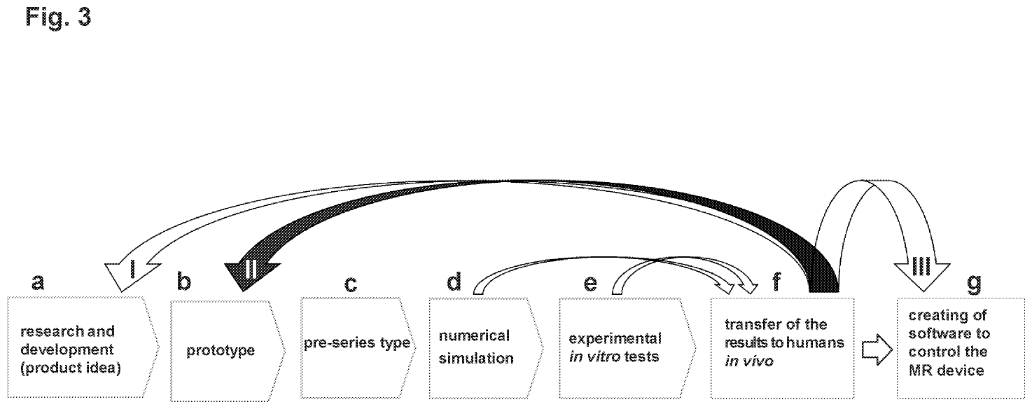

FIG. 3 an example of the process of optimizing implants with the aid of the method according to the invention

FIG. 1 shows the process of the method according to the invention with steps (A) to (E), using a hip implant as an example, on which an E field acts.

FIG. 1A shows schematically the position of the implant IP in the patient PA and the electromagnetic fields EF acting on the implant.

FIG. 1B shows the process flow. In step (A), a computer-generated model CM of the implant is provided, preferably as a CAD (computer-aided design) file. The three-dimensional geometry of the implant is of course known.

Based on the computer-generated model, a one-dimensional representation ER of the implant is defined in step (B) by means of a curve line CT (center trajectory) along the longitudinal axis of the one-dimensional representation ER. The center-trajectory CT preferably runs between the two most distant points and through the axial center of the one-dimensional representation ER. In the case of multiple exposed endpoints, this can be multiple curve lines.

In step (C), followed by step (B), a large number of radii BD (blue disks) are then defined along the center trajectory CT, which enclose the three-dimensional geometry of the implant. At narrow points of the implant, this radius is to be selected correspondingly smaller, at voluminous points the radius is to be selected correspondingly larger. If an implantable object is involved, the blue disks BD should not leave the assumed body tissue.

In the following step (D), a number of auxiliary trajectories HF are via random function laid through the blue disks BD defined in step (C). The set of auxiliary trajectories HT represents the implant with respect to its potential to absorb the applied tangential electric field EF (E-field).

In step (E), the tangential E-field EF at each of the trajectories HT generated in step (D) is evaluated and subsequently statistically processed. The statistics include, for example, the E-field averaged over the individual trajectories, as well as the histographic distribution over the entirety of the trajectories.

The E-field values thus obtained can be used as a realistic mean E-field, which can then be used in subsequent experimental measurement studies.

FIG. 2 shows the proposed classification of the MRT environment into the ranges 0 to 3 and the proposed classification of the objects into the categories i to vi.
Area 0 is located outside the RF-shielded chamber MRK and can be largely excluded for the evaluation of objects with respect to MRT compatibility and MRT safety, since there are hardly any significant interactions between objects in this area and the MRT.
Area I is within the RF-shielded chamber MRK but still outside the currently defined 0.5 mT line and is important for MRT compatibility and MRT safety.
Area II is within the currently defined 0.5 mT line but without significant interference from the RF or gradient field and is important for MRT compatibility and MRT safety.
Area III is located within or in close proximity to the MR bore MRB and thus within the area of influence of both the gradient and RF fields and is important for MRT compatibility and MRT safety.
Category i includes objects in areas I and II such as accessories without patient contact ZB, i.e. shelves, trays, displays, fire extinguishers, etc., which have no contact with the patient.
Category ii includes objects with patient contact in areas I and II, such as wheelchairs, rolators, hypodermic needles, etc., which therefore have direct contact with the patient.
Category iii includes objects such as active and passive implanted medical devices in areas I and II.
Category iv includes objects such as active instruments in area III but without contact with the patient.
Category v includes objects in area III with contact to the patient such as ECG electrodes, invasive measurement catheters, etc.
Category vi includes objects such as active and passive implants IP in area III.

FIG. 3 shows an example of a typical product development process in the field of implant design with the development and research of a product idea a, the creation of a prototype b, the creation of a pre-series model c, the performance of a numerical simulation d, if necessary the performance of additional experimental in vitro tests e, and the transfer of the findings from the numerical and/or experimental data d, e to the human model f.

The essential properties of the implant are determined in particular in steps a and b, i.e. research and development including prototype production. Only when these steps have been satisfactorily completed does the production of ready-to-use pre-series models take place. These models are then subjected to further testing, including computer simulations, which may essentially also involve implant safety in an MR device environment.

In this flow chart, the method according to the invention corresponds to the numerical simulation d for transfer to the human model f. Thus, on the basis of the (processed) data of the numerical simulation d, a decision can be made as to whether the implant is already suitable for use in MRT—i.e. can at best be considered MR-safe—or whether a revision of the implant design must take place.

Accordingly, an optimal design for the corresponding implant can be developed by repeated tests.

The decision as to whether or not an implant meets the requirements is made on the basis of a previously defined limit value.

As described, the heating of an implant in an E-field can be determined using the data of the method according to the invention. If the determined heating exceeds a specified limit value, the implant does not meet the requirements placed on it and must be revised.

In this case, the results of the numerical simulation e may necessitate a complete redesign of the product when transferred to the human model f, which may require a re-entry I also into the underlying research and development a. Alternatively, however, it may only be necessary to re-enter II into the development of an adapted prototype b or an adapted pre-series model c, respectively.

The data from the numerical simulation d provide concrete results as to which areas of the implant are particularly susceptible to damaging heating. Accordingly, the data serve concretely for technical further development and thus have a direct technical effect in the optimization of the implant design.

A corresponding procedural implementation can be performed in a step (F1) after step (E), namely determining the expected heating of the object evaluated according to steps (A) to (E) and comparing the determined heating with a previously determined limit value of the heating.

By specifying certain limit values, it is also possible to derive from the calculated heating whether this heating requires no revision, a complete revision (re-entry according to I) or only a partial revision (re-entry according to II) of the implant.

Accordingly, the data from the numerical method d according to the invention can also result in the decision or finding III that no further changes to the implant are possible, useful or desired. In these cases, the data from the numerical method d according to the invention can also be used to control an MR device during the scan of a patient with an appropriately evaluated implant in such a way that only those sequences of the MRT can be activated for the examination which lead to sufficiently low E-fields and thus ensure the safety of the implant with regard to limited heating. Accordingly, the data of the numerical method d according to the invention directly serve the development of a control system for MRTs, can be integrated into it and thus have a direct technical effect.

A corresponding procedural implementation can be carried out in a step (F2) alternative to (F1) after step (E), namely the use of the data according to steps (A) to (E) to control an MR device, wherein the control only permits those operating modes (sequences) of the MR device that generate an E field that does not reach a critical strength and, in particular, does not cause dangerous heating of the implant.

Thus, given knowledge of the power data of the MR device to be controlled, the data obtained from the numerical method d according to the invention can be used directly to limit the power of the MR device or the usable modes or sequences such that no E-fields critical for heating the implant are generated.

1. Method for evaluating electrically conductive objects comprising the following steps:
(A) Providing a computer-generated model of the object to be evaluated.
(B) Defining the one-dimensional representation of the object by means of a curve line (center trajectory) along the longitudinal axis of the object.
(C) Defining of at least two radii (blue disks) along the center trajectory which enclose the three-dimensional geometry of the object.
(D) Generating a plurality of auxiliary trajectories using the radii defined in step (C), wherein the totalities of the trajectories display the object with respect to its potential to absorb the applied tangential electric field (E-field).
(E) Evaluation of the tangential E-field at each of the trajectories generated in step (D) and subsequent statistical preparation.

2. Method according to claim 1, characterized in that the number of auxiliary trajectories (HT) is between 100 and 1000.

3. Method according to claim 1, characterized in that the center trajectory runs between the two most distant points and through the axial center of the object.

4. Method according to claim 1, characterized in that at least a first radius is provided at a first end of the object and a second radius is provided at a second end of the object.

5. Method according to claim 1, characterized in that the radius of the blue disks is to be selected smaller at narrow locations and larger at voluminous locations, wherein the radii are not leaving the probable body tissue, provided that it is an implantable object.

6. Method according to claim 1, characterized in that the statistics comprise a statement regarding the E-field averaged over the individual trajectories as well as the histographic distribution over the entirety of the trajectories.

7. Method according to claim 1, characterized in that the object is an electrically conductive medical implant selected from the group of active and passive implants.

8. Method according to claim 1, characterized in that the E-field is a high-frequency E-field.

9. Method according to claim 1, characterized in that the E-field is generated by an MR device.

10. Method according to claim 1, comprising a further step (F1) after step (E), namely
(F1) Determining the expected heating of the object evaluated in accordance with steps (A) through (E) based on the data obtained by the method and comparing the determined heating to a predetermined limit of heating.

11. Method according to comprising a further step (F2) after step (E), namely
(F2) Using the data according to steps (A) to (E) to control an MR device, wherein the control only allows such operating modes (sequences) of the MR device that can generate an E-field that does not reach a critical strength and, does not cause dangerous heating of the implant.

12. Computer program executable on a computer, wherein the computer program comprises instructions for performing the method of claim 1.
